(19) United States
(12) Patent Application Publication
(10) Pub. No.: US 2010/0302004 A1
(43) Pub. Date: Dec. 2, 2010

(54) Device and Method for Remotely Monitoring Animal Behavior

(75) Inventors: Chris Winstead, Logan, UT (US); Rebecca Lewis, Logan, UT (US)

Correspondence Address: UTAH STATE UNIVERSITY;TECHNOLOGY COMMERCIALIZATION OFFICE, 570 RESEARCH PARK WAY, SUITE 101, NORTH LOGAN, UT, 84341, US

(73) Assignee: Utah State University, North Logan, UT (US)

(21) Appl. No.: 12/787,498

(22) Filed: May 26, 2010

Related U.S. Application Data
(60) Provisional application No. 61/183,143, filed on Jun. 2, 2009.

(51) Int. Cl. G08B 5/22; A01K 29/00

(52) U.S. Cl. 340/7.32; 119/859; 340/7.2

(57) ABSTRACT

The device and method for remotely monitoring animal behavior consists of a sensor unit mounted on an animal. The sensor unit utilizes motion sensors to monitor the specific behavioral patterns of the animal. Power management is controlled to turn the system on when specific types of motion are detected. Motion data is wirelessly transmitted to a base station for analysis. Data analysis functions are used to detect and identify the occurrence of characteristic motions. Network decision algorithms analyze the characteristic motions and compute weighted indicators and weighted counter-indicators which are combined into a final diagnostic score. When the final diagnostic score exceeds a threshold value, a specific behavioral pattern is confirmed which may indicate the animal is in a distress situation or experiencing another type of behavior that requires intervention. If distress is detected, the system sends a communication to notify the appropriate personnel that a distress condition exists.

### RELATED APPLICATIONS

This application claims priority under 35 U.S.C. §119(e) of U.S. Provisional Patent Application No. 61/183,143, filed Jun. 2, 2009, and titled “Animal Behavior Monitor” and is incorporated herein by reference.

### FIELD OF THE INVENTION

This invention relates to novel sensor systems for continuous monitoring of animals, and more particularly, to the detection and analysis of characteristic motions resulting in animal behaviors that can indicate a variety of activities and conditions, some of which may result in serious injury to the animal.

### BACKGROUND OF THE INVENTION

The actions and movements of an animal can be directly correlated to behaviors that are indicative of trauma, distress, or other potential conditions that may result in serious injury. Colic is the most common cause of premature death in horses. Casting is also a highly common cause of serious injury to equines. Both conditions require immediate intervention by barn staff. If a horse becomes distressed due to colic or casting, and the trauma goes untreated, the condition can result in permanent damage or even death in a matter of hours.

Colic is a term indicating abdominal pain and is one of the most dangerous and costly equine medical problems. Equine colic is a pain experienced by a horse, caused by any number of complications of the intestine. The digestive system of a horse is a complicated series of interactions among many different organs. Equine Colic can originate from the stomach, the small intestine, or the large intestine. The entire digestive network is suspended and nourished by a thin membrane called the mesentery. Colic is associated with any malfunction, displacement, twisting, swelling, infection, or lesion of any part of the equine digestive system.

Different types of colic include the following:

Stomach distention: The small capacity of a horse's stomach makes it susceptible to distension when large amounts of grain are ingested in a single meal. When a horse gorges itself on grain, or a substance which expands when dampened like dried beet pulp, the contents of the stomach can swell. The horse's small stomach and its inability to vomit mean that in these circumstances the stomach may rupture. Once this has happened death is inevitable.

Displacement: The small intestine is suspended in the abdominal cavity by the mesentery and is free floating in the gut. In a “displacement”, a portion of the intestine has moved to an abnormal position in the abdomen. This mobility can predispose the small intestine to become twisted. Except in rare cases, the result is total blockage of the intestine requiring immediate surgery if the horse is to survive. During twisted intestine surgery, the intestine is repositioned and any portion of the intestine that is damaged due to restricted blood flow is removed. Displacement colic can be caused by gas build up in the gut that makes the intestines buoyant and subject to movement within the gut.

Impaction colic: This is the term used when the intestine becomes blocked by a food mass that's too large to easily pass. The large intestine folds upon itself and has several changes of direction (flexures) and diameter changes. These flexures and diameter shifts can be sights for impactions, where a firm mass of feed or foreign material blocks the intestine (including the cecum). Impactions can be induced by coarse feed stuff, dehydration or accumulation of foreign material.

Gas colic: All colics are associated with some gas build up. Gas can accumulate in the stomach as well as the intestines. As gas builds up, the gut distends, causing abdominal pain. Excessive gas can be produced by bacteria in the gut after ingestion of large amounts of grain or moldy feeds. The symptoms of gas colic are usually highly painful but non-life threatening unless untreated, then displacement becomes a possibility.

Spasmodic colic: This occurs due to increased contractions of the smooth muscle in the intestines. These intestinal contractions, or abnormal spasms, cause the intestines to contract painfully. Over excitement can trigger spasmodic colic.

Enteritis/colitis: In some cases the abdominal pain is due to inflammation of the small (enteritis) or large (colitis) intestines. This condition is the result of inflammation of the intestine possibly due to bacteria, grain overload or tainted feed. Horses with enteritis may also have diarrhea. Enteritis is often hard to diagnose and may present itself similar to displacement or impaction colics.

Parasite Infections: Certain types of parasitic infections can cause colic. Strongyles, a dangerous parasitic worm, cause intestinal damage that can restrict blood flow to the intestine. Damage to the walls of the intestine produce a roughened surface that accumulates clots. Other colic producing parasites in horses include ascarids (roundworms) and bot flies which can cause a stomach blockage severe enough to result in colic.

Stress: Travel, herd changes, and schedule disruptions can contribute to stress in horses which may result in colic.

Sand Colic: When fed on the ground in sandy regions (i.e. Florida, Utah, Texas, etc.), sand can accumulate in the horse's cecum. The irritation can cause discomfort, and if there are significant amounts of sand present, the weight can cause the cecum to become displaced.

Colic and casting tend to occur more frequently when a horse is transported or used heavily for competitive events. Colic and casting are especially serious issues with high value horses, which are more likely to be transported for showing, breeding or racing. Colic and casting are indicated by key patterns in a horse's posture, motion, and behavior. A casting horse is stuck on its side or back for an extended period. The reaction of a horse to abdominal pain, such as colic, is typically kicking, rolling, sweating. A colicing horse will roll repeatedly during a short window of time.

At present, there are only a few products based on electronic detection that exist which claim to detect colic, casting, or other distress behavior in animals. These products all have serious shortcomings that prevent them from being used on a large scale. Existing products typically originated as foaling monitors, and now claim colic detection as a secondary function. Because of their history as foaling monitors, these products are designed for monitoring a single horse, which is ready to foal, only for a short period of time.

One existing foaling alarm system contains a sensor that is attached to the horse with a belly band. The sensor and wireless transmitter are housed within a radio tower on the horse's back. In practice, horses tend to become preoccupied with nipping at belly bands, making it a distraction for horses and staff. The belly band also introduces a new injury risk due to the transmitter unit mounted on the horse's back. A severely colicing horse is likely to roll over the unit, which may result in a back injury. The methods for mounting this, and other existing foaling sensors, and the position of these mounted sensors on a horse, are not optimal for colic detection. The typical behavior of a colicing horse can result in damage to the sensor and injury to the horse.

A few products use simple radio frequency (RF) transmitters to signal an alarm when a mare lies down for foaling. These alarms appear to consist of a tilt sensor and timer. When the horse stays down for a specified period of time, the alarm is activated. These systems can be connected to a phone line or pager to automatically alert barn staff. These systems use single-channel RF modulation to transmit sensor data. Because multiple transmitters interfere with each other, the RF approach cannot be scaled up for large operations with many horses. Interference can also arise from other RF transmitters, such as cordless phones or even a neighbor's foaling alert device. Hence these products are best suited for small farms.

Given the deficiencies of the technologies cited above, barn managers currently use round-the-clock night checks by barn staff to monitor the health and safety of their horses. Manual checks are costly, and are not fool-proof. Even with individuals on location twenty-four hours a day in a veterinary facility or racing barn, signs of trouble might not be caught as early as desired. A safe, reliable, and unobtrusive animal behavior monitor system, which operates continuously and can monitor more than one animal simultaneously, and alert staff as soon as there are signs of distress, is needed.

### SUMMARY OF THE INVENTION

Early detection of animal distress, such as colic in horses, leads to prompt treatment, which can vastly increase the animal's chance of survival. Due to the high cost of surgery, it is especially desirable that the animal receives this treatment (when necessary) at the first signs of colic in order to minimize the risk of mortality and the sunk costs of a failed surgery. Thus, a monitoring system that employs a motion sensor attached to the horse's halter, and relays tell-tale signs of colic to a receiver is indispensable in safeguarding horses—without the need for constant human presence.

The device and method disclosed herein consist of a sensor unit for measuring characteristic motions of an animal, and a computational process for the automatic analysis of the motions to identify specific behaviors. The device can be adapted to monitor behavior patterns of a wide variety of animals such as, but not limited to, cattle, elk, llama, bison, bears, sheep, deer, etc.

One wide ranging application for the device is the monitoring of horses. The sensor detects characteristic motions, which are automatically analyzed and associated with behaviors that may indicate colic, casting or other serious conditions that require immediate intervention. The sensor unit contains a three-axis accelerometer. The accelerometer output is correlated with coarse posture information which is used to indicate a possible distress situation. If the sensor unit detects motions associated with rolling or casting for an unusual period of time, it wirelessly transmits fine-scale accelerometer data to the base station computer.

The base station computer evaluates a variety of fine-scale heuristic indicators that may prove or rule out distress. The base station uses network decision algorithms to analyze the animal's behavior and decide whether intervention is needed. If a distress situation is detected, the base station alerts barn staff utilizing any of a variety of electronic communication methods. In addition, a veterinary service, or other qualified organization can be contacted directly.

The device may be implemented to continuously monitor horses in locations such as stalls, pastures, breeding centers, show barns and veterinary facilities, as well as in trailers or other modes of transportation. When distress is detected, the device may relay the emergency situation to appropriate barn staff, veterinary personnel, owners, etc. via wireless communication methods. Various research applications are also enabled by the device, such as identifying more subtle equine conditions based on behavioral signatures, and monitoring the behavior of wild horse herds.

### BRIEF DESCRIPTION OF THE DRAWINGS

Understanding that drawings depict only certain preferred embodiments of the invention and are therefore not to be considered limiting of its scope, the preferred embodiments will be described and explained with additional specificity and detail through the use of the accompanying drawings in which:

FIG. 1 shows the sensor unit attached to the halter on top of a horse's head.

FIG. 2 illustrates the use of the device and range extenders in a horse pen or pasture containing more than one horse.

FIG. 3 illustrates the use of range extenders to monitor a large facility with numerous stalls, pens and pastures.

FIG. 4 shows one potential single case configuration of the components in the device.

FIG. 5 illustrates the double case non-protruding packaging of the components that can lie flat against a horse.

FIG. 6 is a sensor unit flow chart decision diagram illustrating one embodiment of a method for power management and obtaining motion data.

FIG. 7 is a functional block diagram of a microcontroller configured to manage the sensor unit.

FIG. 8 is a base station computer decision diagram illustrating one embodiment of a method for analyzing behavioral motions and determining if they indicate a distress event.

FIG. 9 is an example of a plot showing the output from the roll and shake analysis functions.

FIG. 10 is an example of a plot showing the indicator score, counter-indicator score, and final diagnostic score of a possible distress event

FIG. 11 is a functional block diagram of a computer system configured to process and analyze sensor data.

### DETAILED DESCRIPTION OF SELECTED EMBODIMENTS

In the following description, numerous specific details are provided for a thorough understanding of specific preferred embodiments. However, those skilled in the art will recognize that embodiments can be practiced without one or more of the specific details, or with other methods, components, materials, etc. In some cases, well-known structures, materials, or operations are not shown or described in detail in order to avoid obscuring aspects of the preferred embodiments. Furthermore, the described features, structures, or characteristics may be combined in any suitable manner in a variety of alternative embodiments. Thus, the following more detailed description of the embodiments of the present invention, as represented in the drawings, is not intended to limit the scope of the invention, but is merely representative of the various embodiments of the invention.

Much of the infrastructure that can be used with embodiments disclosed herein is already available, such as: general purpose computers; computer programming tools and techniques; and digital storage media. A computer may include a processor such as a microprocessor, microcontroller, logic circuitry, or the like. The processor may include a special purpose processing device such as an ASIC, PAL, PLA, PLD, Field Programmable Gate Array, or other customized or programmable device. The computer may also include a computer readable storage device such as non-volatile memory, static RAM, dynamic RAM, ROM, CD-ROM, disk, tape, magnetic, optical, flash memory, or other computer readable storage medium.

Aspects of certain embodiments described herein are illustrated as software modules or components. As used herein, a software module or component may include any type of computer instruction or computer executable code located within a computer readable storage medium. A software module may, for instance, comprise one or more physical or logical blocks of computer instructions, which may be organized as a routine, program, object, component, data structure, etc., that performs one or more tasks or implements particular abstract data types.

In certain embodiments, a particular software module may comprise disparate instructions stored in different locations of a computer readable storage medium, which together implement the described functionality of the module. Indeed, a module may comprise a single instruction or many instructions, and may be distributed over several different code segments, among different programs, and across several computer readable storage media. Some embodiments may be practiced in a distributed computing environment where tasks are performed by a remote processing device linked through a communications network. In a distributed computing environment, software modules may be located in local and/or remote computer readable storage media. In addition, data being tied or rendered together in a database record may be resident in the same computer readable storage medium, or across several computer readable storage media, and may be linked together in fields of a record in a database across a network.

The software modules described herein tangibly embody a program, functions, and/or instructions that are executable by computer(s) to perform tasks as described herein. Suitable software, as applicable, may be readily provided by those of skill in the pertinent art(s) using the teachings presented herein and programming languages and tools, such as XML, Java, Pascal, C++, C, database languages, APIs, SDKs, assembly, firmware, microcode, and/or other languages and tools.

Disclosed are embodiments of a novel animal behavior monitoring system, in the form of a wearable sensor unit, range extender units, and a base station computer, that analyzes motion data associated with the health and safety of animals. The animal behavior monitoring device includes sensors, a power source, analysis algorithms, and communication capabilities for the long term monitoring of the behavior of many animals simultaneously. The embodiments described herein are presented within the context of equines, and the animal behavior monitoring device will be referred to as an Equine Distress Monitor (EDM), although it should be obvious to one skilled in the art the device is applicable to a host of different animals under myriad conditions. Equine health issues, such as colic and casting are indicated by key patterns in a horse's behavior. A casting horse is stuck on its side or back for an extended period. A colicing horse will roll repeatedly during a short window of time. The EDM detects the above patterns by continuously monitoring the horse's posture and movements.

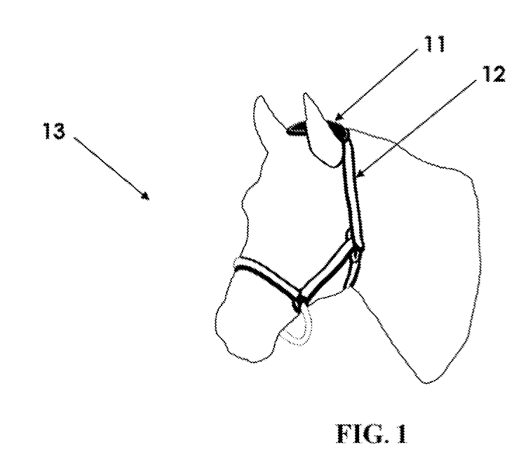

An Equine Distress Monitor sensor unit is a small case containing sensor and electronic components mounted on each horse to monitor its behavior around the clock, without requiring extra supervision or effort from barn staff. As illustrated in FIG. 1, the wearable EDM sensor unit 11 is attached to the halter 12 on top of the head of the horse 13. In different embodiments the sensor can be attached to the collar, surcingle, or other tack or equipment as appropriate. For other animals, the animal behavior monitoring device can be attached to the animal by methods such as ear tags, harnesses, ankle bands, tail mounts, or other appropriate techniques.

The sensor unit's unobtrusive design and long battery life make it a safe solution for routine, long-term monitoring of animal health and safety, and is suitable for adoption in large-scale operations such as breeding centers, show barns and racing barns. Given the fact that in the preferred embodiment the horse's neck movements are critical to the functionality of the system, the sensor unit is designed to fit into a small, relatively flat package that attaches to a break-away collar or halter. In one embodiment the sensor's double case enclosure bends to follow the contour of the horse's head or neck. In other embodiments the sensor's components fit in a single small case. The EDM's small form factor is a key feature that makes it acceptable for routine long-term use in a large barn. The device does not pose any risk of snagging on fences, feeders or other objects. The EDM attaches to a break-away halter to avoid snagging or hanging up. The device does not protrude or have an unusual appearance that may attract the curiosity of other horses. The EDM sensor unit lies flat against the horse within a leather or nylon case, so that it looks, feels, and tastes like any other barn object.

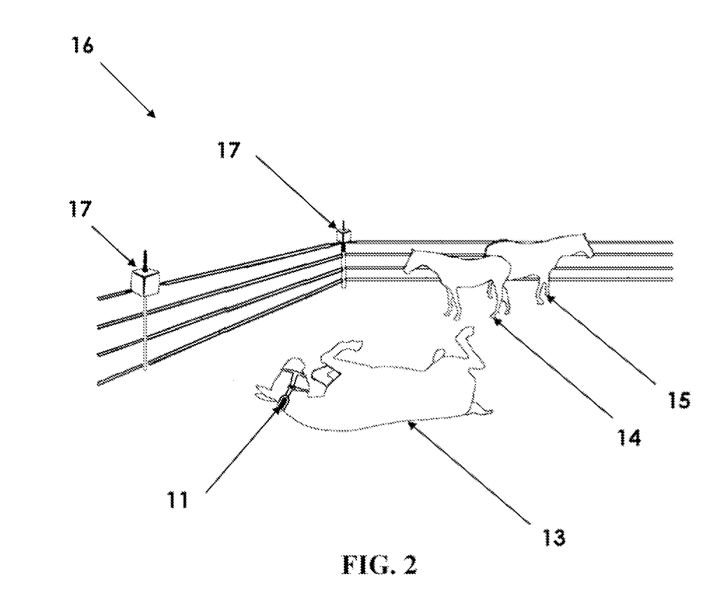

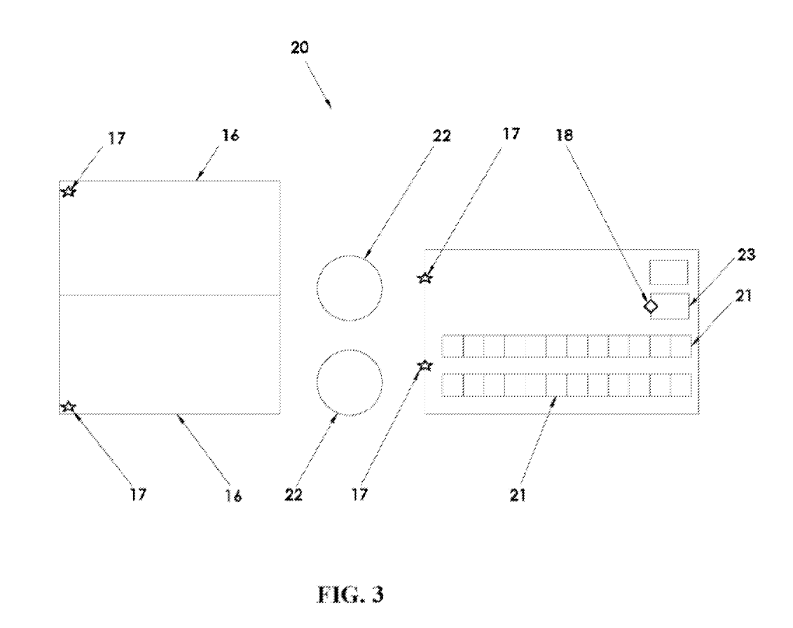

FIG. 2 is an illustration of the EDM sensor unit 11 in use in a horse pen or pasture 16. Every horse 13, 14, 15 is wearing an EDM sensor unit 11. (The sensor unit is not visible on horses 14 and 15 in FIG. 2). When one of the horses 13 begins to roll, the sensor unit 11 wirelessly transmits motion data to the base station computer (not shown). The data is routed through an ad-hoc network of range extender units 17. FIG. 3 illustrates the implementation of the EDM system at a large equine facility 20. The range extender units 17 allow for the coverage of a large equine facility 20 with numerous stalls 21, pens 22 and pastures 16. In this example, range extender units 17 are placed on the fences in the pastures 16 and on either side of the barn buildings, where they provide a bridge between indoor and outdoor signal paths. The range extender units 17 route the data to the base station computer 18 which is generally located in an office 23 of the facility 20.

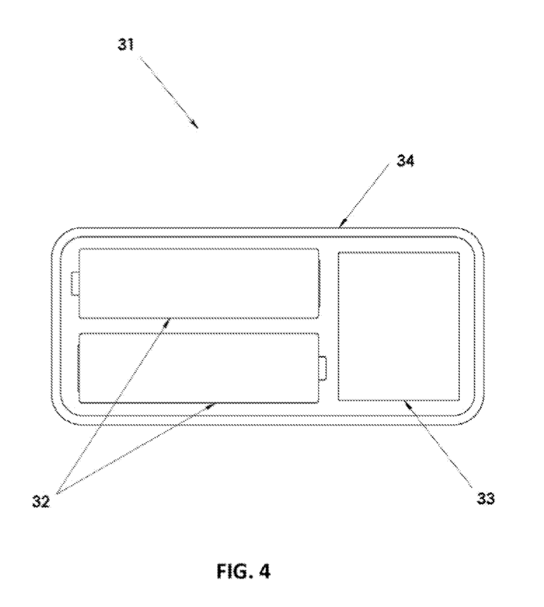

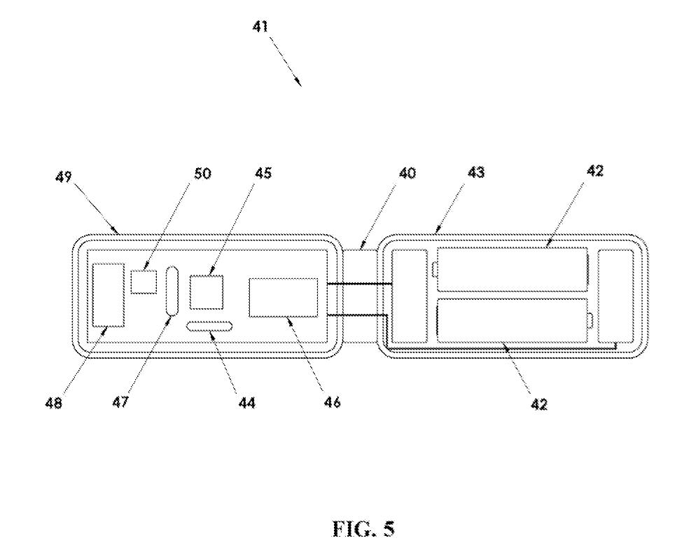

One embodiment of the EDM system comprises the sensor unit, which houses a microcontroller, a wireless transceiver, a tilt sensor, an accelerometer, a unique identifier chip, associated electronics, and batteries, and additional components comprising range extenders and a base-station computer. An optional programming interface can be included. The individual devices can be arranged in the sensor case in a variety of configurations. FIG. 4 shows a single case EDM sensor unit 31 and FIG. 5 shows a double case with a flexible joint EDM sensor unit 41.

One example of the single case EDM sensor unit 31, shown in FIG. 4, comprises the batteries 32 and electronic devices 33 within a single case 34 or housing with dimensions on the order of 9 cm×5 cm×1.5 cm. The electronic devices 33 comprise a tilt sensor, an accelerometer, a programmable microcontroller, a wireless transceiver, a unique identifier chip, and a programming interface.

One example of the double case with a flexible joint EDM sensor unit 41, shown in FIG. 5, comprises the batteries 42 in a first case 43 or housing with dimensions on the order of 6 cm×4 cm×1.5 cm. The electronic devices comprising a tilt sensor 44, an accelerometer 45, a programmable microcontroller 46, a wireless transceiver 47, a unique identifier chip 50, and a programming interface 48 are located in a second case 49 or housing also with dimensions on the order of 6 cm×4 cm×1.5 cm. The first case 43 is attached to the second case 49 by a flexible coupling 40 which contains electrical connections between the batteries and the electronics.

The microcontroller is programmed to analyze and control the functions of the electronic devices in the EDM sensor unit. The tilt sensor is used for power management. The accelerometer is a three-axis sensor device, which provides coarse equine posture information (e.g. the sensor's tilt angle in three dimensions), as well as fine motion information (e.g. trotting, shaking off, or struggling). In other embodiments inertial sensors can be used in addition to, or in place of accelerometers. The transceiver is the basis for wireless network communications of the distress indicators. The programming interface is used for loading program instructions into the controller. In another embodiment, the programming interface may be eliminated because the controller can be programmed prior to component assembly.

Power management of the EDM sensor unit is critical for long term low maintenance operation of the system. The sensor unit remains active for a set period of time, and then shuts itself off again. The device uses high-capacity, low self-discharge batteries, such as, or similar to NiMH batteries. These batteries allow the device to sit idle for hours, days or even months without losing significant battery charge.

In addition, wireless data transmission is carefully managed to conserve power. Algorithms in the sensor are used to associate animal motion with specific behaviors of interest. Data is only transmitted when certain actions, such as possible coarse distress behaviors are observed. The sensor units communicate with the base station, and can also communicate with range extending devices, or even with each other. Because the sensor's power consumption is tightly managed, the batteries need to be replaced infrequently.

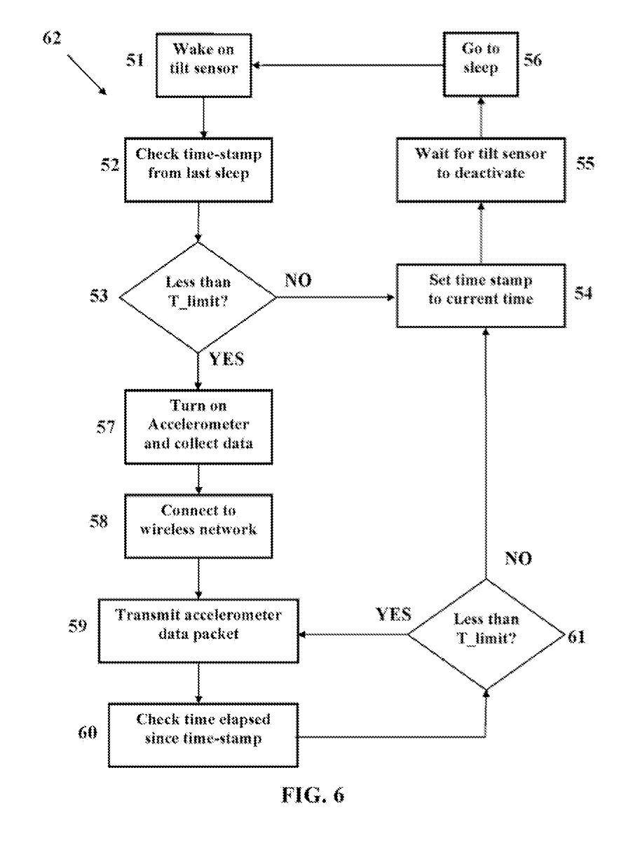

The microcontroller is designed with a micro-power sleep mode and wake-on-signal operation. The microcontroller is in sleep mode most of the time, requiring essentially no power. The microcontroller responds to the tilt sensor according to the sensor unit flow chart decision diagram 62 in FIG. 6. A mechanical tilt sensor causes the microcontroller to wake 51 and begin operation when the sensor is rotated greater than a predetermined value, such as when the horse lies on its side. The current wake time stamp is compared with the previous time stamp from the last sleep 52 and if the time difference is not within a designated time period 53, the time stamp is set to the current time 54, the tilt sensor is deactivated 55, and the sensor unit goes back to sleep 56. This power management loop is essentially a coarse false alarm check.

An indicator that a potential distress event is occurring is characterized by the microcontroller waking repeatedly within a designated time period 53 causing the system to go into the operational mode. In this situation, the accelerometer is turned on 57 and it begins collecting motion data, and the transceiver is activated, meaning the devices are powered up and the sensor unit is connected to the wireless network 58. Data from the accelerometer is transmitted 59 to the base station computer to provide motion data to be analyzed for a possible distress or colic event. The time elapsed since the previous time stamp 60 is determined and if this is within a designated time-period 61, the sensor unit continues to transmit accelerometer data 59 to the base station computer. Once data has been transmitted to the base station computer for a predetermined time period, i.e. the elapsed time since the last time stamp is outside the designated time period 61, the system sets the time stamp to the current time 54, the tilt sensor is deactivated 55, and the sensor unit, including the accelerometer and transceiver, goes back into the sleep mode 56.

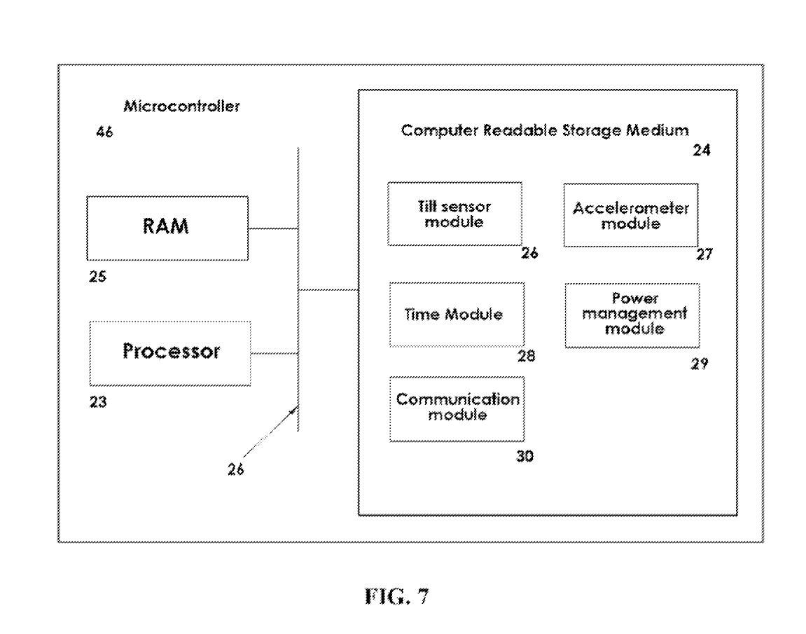

FIG. 7 illustrates one possible implementation of a system that may be utilized to perform the method of FIG. 6. The microcontroller 46 includes a processor 23, a computer readable storage medium 24, Random Access Memory (RAM) 25, and a bus 26. The bus 26 provides a connection between the RAM 25, the processor 23, and the computer readable storage medium 24. The processor 23 may be embodied as a general purpose processor, an application specific processor, a digital signal processor, or other device known in the art. The processor 23 performs logical and arithmetic operations based on program code stored within the computer readable storage medium 24.

The computer readable storage medium 24 may comprise various modules for analyzing power and motion data. Such modules may include a tilt sensor module 26, an accelerometer module 27, a time module 28, a power management module 29, and a communication module 30. Each module may perform one or more tasks associated with powering up and powering down the sensor unit plus collecting and transmitting accelerometer data to the base station.

The base station computer can be an ordinary, general purpose computer, a hand held personal computer, or any type of system hardware with the appropriate computing capabilities. It is connected to a wireless network, capable of communicating with the EDM sensor unit devices. In its default state, the base station waits to receive data from the sensor network. Each sensor unit on the network is provided with a unique identifier (UID), so that the base station can distinguish sensor information from many individual horses. For each UID, a unique memory range is assigned for storing the corresponding accelerometer data.

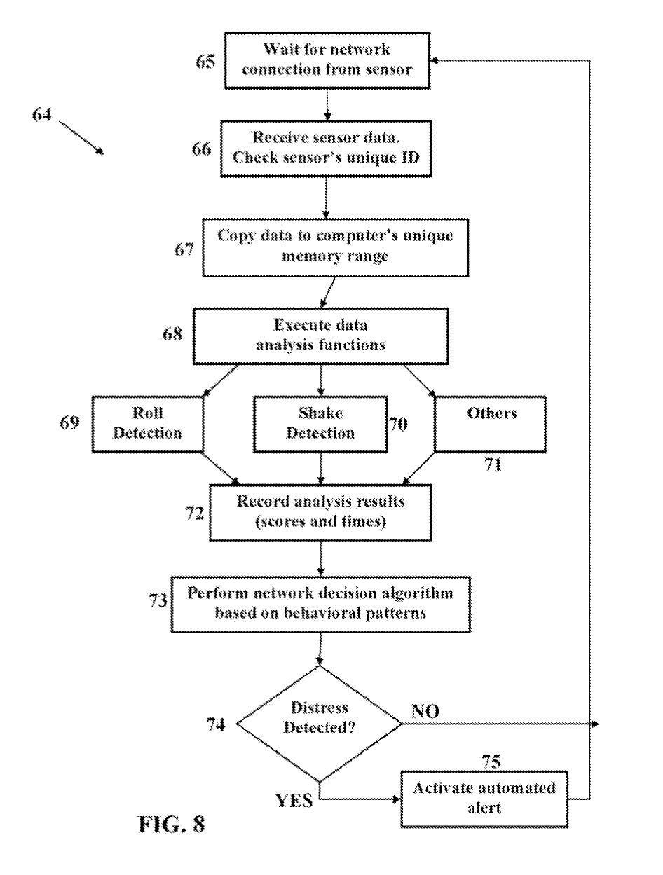

Once a packet of accelerometer data is received, the base station responds according to the base station computer decision diagram 64 shown in FIG. 8. When the sensor unit wakes, the wireless network is connected with the base station computer 65. The base station computer receives accelerometer sensor data and checks its UID 66. The received accelerometer data is copied into the computer's unique memory range 67, and the data analysis functions are executed 68. The data analysis functions use pattern recognition algorithms to filter and analyze the motion data. The purpose of these data analysis functions is to detect and identify the occurrence of specific characteristic motions, including rolling 69, shaking 70, and others 71 such as trotting, etc.

Each characteristic motion may be an indicator of a distress condition, or a counter-indicator. Positive equine distress indicators include repeated rolling with short time intervals between rolls, nipping at sides, and high activity over an extended time period while the horse is lying on its side. Counter equine distress indicators include shaking off after rolling, low activity with a brief duration while the horse is lying on its side, and very little to no motion at all.

The data analysis functions 68 are a set of algorithms used to compute an estimate of the probability of a designated characteristic motion at any specific time, such as rolling or shaking. For example, the function detect roll analyzes the most recently received accelerometer data, and produces a probability Pr(a roll occurred;t). This probability may be estimated in a variety of ways. In one embodiment, the accelerometer data is first converted into spherical coordinates consisting of a tilt angle (Ø), an incline angle (θ), and an intensity (r). Given the spherical data, there are several possible ways to estimate a characteristic motion. Examples of two methods for roll motion detection are:

1) Binary estimator: The spherical data is first low-pass filtered using a simple averaging-smoothing operation. The tilt angle is then monitored. If Ø>0 at time t, but Ø<0 at time t+1, then a roll is detected at time t+1. The function's output is ‘1’ at time t+1 and ‘0’ at other times.

2) Correlation estimator: The spherical data is correlated with a suitable mask pattern. The correlation output, y(t) is then passed through a parameterized sigmoid function, which produces an output between zero and one. An example sigmoid function is

$$s ( y ) = \frac{{e}^{( α ( y - μ ) )}}{1 + {e}^{( α ( y - μ ) )}} .$$

In this function, the parameter μ defines the threshold for detecting a roll (i.e. the value of y for which s=0.5). The parameter a sets the sharpness of the threshold. The values of μ and α are calibrated empirically using field data from observed roll and non-roll events.

A sequence of characteristic motions over time forms a behavioral pattern. With continuing reference to FIG. 8, the data analysis provides quantitative characteristic motion results expressed as scores and times 72. These scores and times are associated with specific behavioral patterns and are used to diagnose distress conditions via a network of decision algorithms 73. The timing and order of behaviors strongly influences the diagnosis.

The network decision algorithm 73, described below, can be a basic expert diagnosis algorithm for the identification of behavior associated with a distress condition. A collection of data analysis functions returns a set of indicators and counter-indicators s₁(t), s₂(t), s₃(t), . . . Each indicator or counter-indicator is multiplied by a weight wᵢ factor (usually less than one). The weighted indicators are summed and integrated over time to obtain an overall score. Repeated indicator events increase this score. The integration also includes a decay parameter, δ(t), so that the score decreases after sufficient time has passed. The decay parameter is chosen so that the score drops to zero after some designated time interval once the indicator events have ceased. Indicators and counter-indicators are processed separately, and are then combined in such a way that the final diagnostic score is increased by the indicators and decreased by the counter-indicators.

An example implementation of the network decision algorithm 73 follows. Let C be the event that a horse has colic, and let C be the event that the horse does not have colic. An indicator filter estimates the probability that the horse has colic, given the particular characteristic motion monitored by that filter. As a minimal example, consider an implementation with two filter algorithms, A₁ and A₂, which provide the following data:

s₁(t)=Probability that a roll occurred at time t.

s₂(t)=Probability that a shake occurred at time t.

y₁(t)=Pr(C, given indicators).

y₂(t)=Pr(C, given counter indicators).

Based on these definitions, the final score can be computed as

D(t)=y₁(t)(1−y₂(t)),

which can be stated as “the probability that there is colic given the indicators (first term), and that colic is not excluded by the counter-indicators (second term)”.

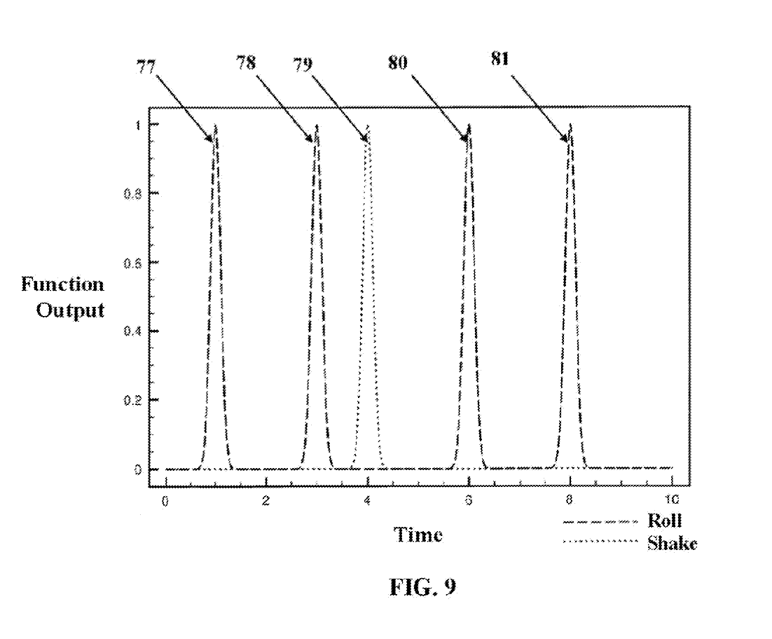

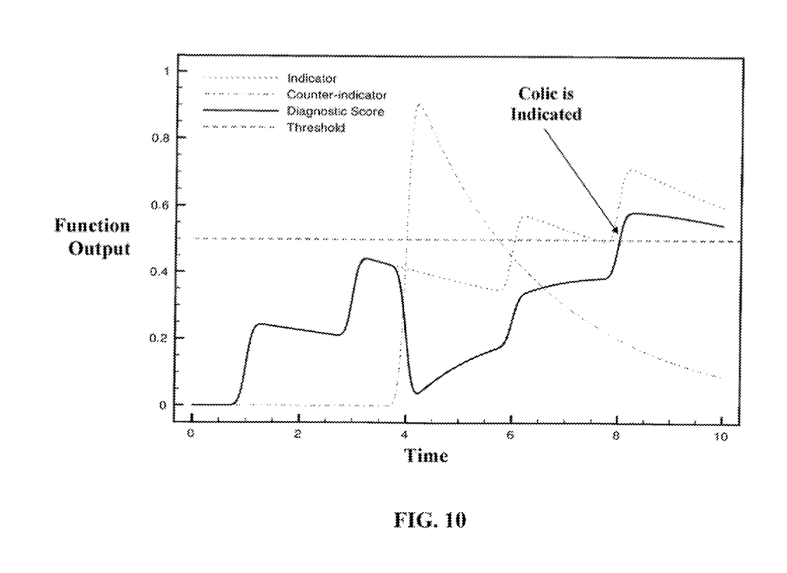

FIG. 9 shows an example behavior pattern reported by the analysis functions (step 68 in FIG. 8). This figure indicates that there is a first roll 77 and a second roll 78, followed by a shake 79, followed by a third roll 80 and a fourth roll 81. The roll and shake data are separately weighted and integrated, yielding the indicator and counter-indicator scores plotted in FIG. 10. In this example, the weight w₁ is chosen so that colic is indicated when there are three or more rolls in a 10-minute interval. The weight w₂ is chosen so that a single shake brings the diagnostic score effectively to zero. The roll indicators have a half-life of five minutes. The shake counter-indicators have a half-life of two minutes.

Once the diagnostic score crosses the threshold of 0.5, as shown in FIG. 10, it is concluded that colic is more strongly indicated than counter-indicated. Referring back to FIG. 8, when the decision algorithm result is greater than a threshold value 74 distress is detected. At this point, the animal should be examined by a knowledgeable person. The automated alert function is activated 75 to notify the appropriate personnel that a distress condition exists.

Because the data is associated with a UID, multiple animals can be monitored simultaneously and the individual animal experiencing the distress is identified. The base station computer alerts barn staff utilizing any of a variety of electronic communication methods. If the decision algorithm results are less than a threshold value 74 the base station computer waits for a future connection from the EOM sensor 65.

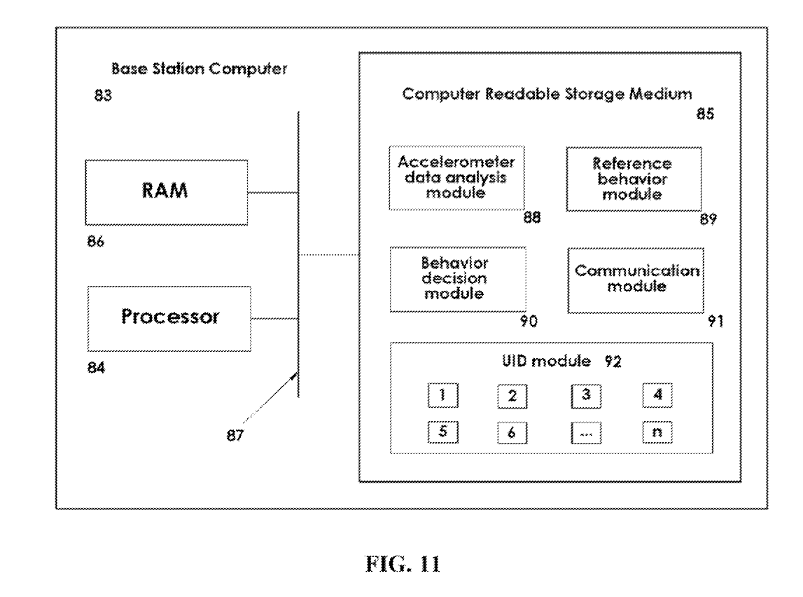

FIG. 11 illustrates one possible implementation of a system that can be utilized to perform the method of FIG. 8. The base station computer 83 includes a processor 84, a computer readable storage medium 85, Randon Access Memory (RAM) 86, and a bus 87. The bus 87 provides a connection between the RAM 86, the processor 84, and the computer readable storage medium 85. The processor 84 may be embodied as a general purpose processor, an application specific processor, a digital signal processor, or other device known in the art. The processor 84 performs logical and arithmetic operations based on program code stored within the computer readable storage medium 85.

The computer readable storage medium 85 may comprise various modules for analyzing motion and behavioral data. Such modules may include an accelerometer data analysis module 88, a reference behavior module 89, a behavior decision module 90, a communication module 91, and a set unique identifier modules 92. Each module may perform one or more tasks associated with receiving motion data, identifying specific behaviors, evaluating distress conditions, and communicating the occurrence of events.

While specific embodiments and applications have been illustrated and described, it is to be understood that the disclosed invention is not limited to the precise configuration and components disclosed herein. Various modifications, changes, and variations apparent to those of skill in the art may be made in the arrangement, operation, and details of the device and methods of the present invention disclosed herein without departing from the spirit, scope, and underlying principles of the disclosure.

We claim:

1. A method for remotely monitoring and characterizing animal behavior, the method comprising:
mounting a sensor unit on an animal;
said sensor unit generating motion data;
wirelessly transmitting said motion data to a computer;
said computer executing data analysis functions to detect and identify the occurrence of characteristic motions; and
said computer executing a network decision algorithm to identify specific behavioral patterns.

2. The method of claim 1 further comprising:
maintaining said sensor unit in a default micro-power sleep mode;
waking said sensor unit after a tilt is detected;
turning on accelerometer;
collecting motion data with said accelerometer;
connecting to wireless network;
transmitting said motion data over said wireless network;
repeating said data collecting and transmitting steps as long as said tilt is detected; and
returning said sensor unit to said default micro-power sleep mode after said tilt is no longer detected.

3. The method of claim 1 wherein executing data analysis functions further comprises converting said motion data into spherical coordinates and using pattern recognition algorithms to analyze and filter said motion data to compute an estimate of the probability of a designated motion.

4. The method of claim 3 wherein said estimate of the probability of a designated motion is computed using a binary estimator function.

5. The method of claim 3 wherein said estimate of the probability of a designated motion is computed using a correlation estimator function.

6. The method of claim 1 wherein executing said network decision algorithm further comprises:
obtaining a set of indicators from said data analysis functions;
obtaining a set of counter-indicators from said data analysis functions;
multiplying said indicators by a first weight factor to obtain a weighted indicator;
multiplying said counter-indicators by a second weight factor to obtain a weighted counter-indicator;
summing said weighted indicators, integrating over time, and factoring in a first decay parameter to obtain an overall indicator score;
summing said weighted counter-indicators, integrating over time, and factoring in a second decay parameter to obtain an overall counter-indicator score;
combining said overall indicator score and said counter-indicator score to compute a final diagnostic score.

7. The method of claim 6 further comprising establishing a threshold value such that when said final diagnostic score exceeds said threshold value, a specific behavioral pattern is confirmed.

8. The method of claim 1 wherein said sensor unit is configured with an identification tag and said transmitted motion data is identified by, and can be traced to, said identification tag.

9. A method for remotely monitoring multiple animals and characterizing individual animal behavior, the method comprising:
mounting a sensor unit with a unique identifier on each individual animal;
programming each said sensor unit to activate an accelerometer within said sensor unit when triggered by a tilt function, and deactivate said accelerometer after a period of time with no tilt activity;
collecting motion data from said accelerometer when activated;
transmitting said motion data with said unique identifier to a computer;
evaluating said motion data using analysis functions programmed within said computer to identify characteristic motions of said animal; and
correlating said motion data with said unique identifier to determine which animal experienced said characteristic motions.

10. The method of claim 9 wherein evaluating said motion data using analysis functions further comprises;
converting said motion data into spherical coordinates;
using pattern recognition algorithms to filter and analyze said motion data; and
computing an estimate of the probability of a designated motion.

11. The method of claim 10 wherein said estimate of the probability of a designated motion is computed using a binary estimator function.

12. The method of claim 10 wherein said estimate of the probability of a designated motion is computed using a correlation estimator function.

13. The method of claim 9 wherein said characteristic motions are further analyzed by executing a network decision algorithm to identify specific behavioral patterns.

14. The method of claim 13 wherein executing said network decision algorithm further comprises:
obtaining a set of indicators from said data analysis functions;
obtaining a set of counter-indicators from said data analysis functions;
multiplying said indicators by a first weight factor to obtain a weighted indicator;
multiplying said counter-indicators by a second weight factor to obtain a weighted counter-indicator;
summing said weighted indicators, integrating over time, and factoring in a first decay parameter to obtain an overall indicator score;
summing said weighted counter-indicators, integrating over time, and factoring in a second decay parameter to obtain an overall counter-indicator score;
combining said overall indicator score and said counter-indicator score to compute a final diagnostic score.

15. The method of claim 14 further comprising establishing a threshold value such that when said final diagnostic score exceeds said threshold value, a specific behavioral pattern is confirmed.

16. A device for monitoring animal behavior comprising:
a housing capable of being mounted unobtrusively on an animal;
a tilt sensor within said housing;
a three axis accelerometer within said housing;
an electronic module with microcontroller within said housing;
a wireless transceiver within said housing;
a battery within said housing;
whereas said housing and components within are collectively referred to as a sensor unit; and
a computer with data analysis and decision algorithm software.

17. The device of claim 16, further comprising a power management module within said microcontroller programmed to activate and deactivate said accelerometer and said transceiver upon motions of said tilt sensor within a specified time period.

18. The device of claim 16, further comprising one or more range extender units to receive and transmit data collected by said accelerometer.

19. The device of claim 16, further comprising an accelerometer data analysis module within said computer to execute data analysis computations to identify the occurrence of characteristic motions.

20. The device of claim 16, further comprising a behavior decision module for executing a network decision algorithm to identify and confirm specific behavioral patterns.
